(19) United States
(12) Patent Application Publication
(10) Pub. No.: US 2010/0041129 A1
(43) Pub. Date: Feb. 18, 2010

(54) Device and system for the installation of an air filtration system in an existing incubator

(76) Inventor: Michael D. Cecchi, Madison, CT (US)

Correspondence Address: William W. Jones, 6 Juniper Lane, Madison, CT, 06443, US

(21) Appl. No.: 12/228,548

(22) Filed: Aug. 14, 2008

(51) Int. Cl. C12M 1/12; B01D 50/00

(52) U.S. Cl. 435/297.5; 55/385.2

(57) ABSTRACT

An improved air purification system is added to an existing incubator or environmental chamber. The improved air purification system includes a series of parts, such as a plate, motor box, power connections and filter to be retrofitted and/or installed into an available interior space of the incubator. The system increases the effectiveness of collecting and removal of particulates and VOC's for the internal environment of the incubator or environmental chamber, without expensive or cumbersome design alterations. The system may be installed at the factory or later on once the incubator or environmental chamber is in use. This will allow an alternative air purification system into existing incubators which will result in better results.

### TECHNICAL FIELD

This invention relates to an apparatus and system for installing a CODA air filtration system into an existing incubator or environmental chamber, without incurring large redesign costs and costly alterations to the original configuration of the incubator or filter. The apparatus and system utilizes current space restrictions and becomes a integral part of the air filtration within the incubator. The invention allows the retrofitting of the system into incubators, overcoming inherent design problems of the incubators and the inability to alter their design after final production. The system may be used in a varied kinds and brands of incubators, environment chambers. The apparatus in some cases includes the modification of an existing system within the incubators to allow the current air flow to not be impeded and the CODA filter system to maximize the clean air flowing inside the incubators. The incubators are strategic in the culturing and growth of oocytes, embryos, stem cells and the like.

### BACKGROUND ART

Currently incubators and environmental chambers are built to existing specifications and methods. The incubators and chambers are built by welding stainless steel pieces together and then adding electronics. Many of these incubators are the same shape, size involving the same methods of manufacturing that have been going on for years.

These units may already have a filtration system in them, either a HEPA filter or combination HEPA and VOC filter. The filters are generally installed in the top portion of the units and are attached to the shield that may form a part of the interior shelving system, or are independently installed.

The incubators are made of stainless steel, and most have water jackets surrounding the interior for the warming of the interior. These designs do not allow the design to be readily changed nor does it allow any alterations to the incubators once they have been produced. Very little can be done to the incubators once they have been delivered for use or are in use.

These systems have very little ability to change the internal configuration as the manufacturers do not wish to incur the added costs of redesigning the current models. These systems are restricted where the filtration devices may be placed inside the incubator.

Small incubator of less than 2 cu. ft have little space to install any type of filtration system and larger incubators of around 6 cu. ft. lack space and will require the invention to be placed on a self, which would take up a large amount of shelf space and the customers are not prone to do so and to give up shelf space.

The problem continues of installing a CODA air filtration system inside an exiting incubator, as the air filtration system contains many parts. A motor for moving the air, a filter and filter attachments connected by air tubing and held in place with a shield or box. The many parts and placement of the parts make the installation difficult and the placement becomes important to the space in the incubator and its ability to conserve space, as not to take up too much useful space in the incubator. Details of the construction and operation of the CODA filter system can be found in U.S. Pat. No. 6,200,362, which is incorporated herein in its entirety.

It would therefore be highly desirable to provide an apparatus which will be able to be retrofit the CODA air filtering assembly into existing incubators to improve the air filtration of that incubator so as to remove particulates as well as volatile organic compounds from the air in the incubator.

### DISCLOSURE OF THE INVENTION

This invention relates to an improved installation and support device which is designed to retrofit CODA filtering assemblies into current incubators, and to be able to add the CODA assemblies to newer incubators without expensive modifications to the incubator itself. The incubators are used in the culturing of mammalian cells such as oocytes, embryos, stem cells and other specimens. The incubators can be used in animal as well as insect culturing.

The installation and support device is designed to be retrofitted into older incubators, or positioned in the newer incubator models in many variations, depending on the space available and the configuration of the incubator.

One embodiment of the support device is designed for placement in the head space of an older incubator. The support device can use the existing support pins or support brackets of the incubator. The support device is preferably made of stainless steel and is able to utilize the incubator pin locations in the design. The support device will also take into consideration the placement of current fans and sensor devices which have been installed in the upper roof portion of the incubator. In this embodiment, the support device must contain slots for the pin placement, and the slots must settle on these pins to hold the support device in place. The support device must also have an open vented area cut therein to allow the air flow generated by the existing fan in the incubator roof. The support device must be configured to allow the sensors in the roof to be unimpeded and be able to work at peak efficiency. The support device must also contain mounting holes for the incubator air circulation motor, and openings for the mounting of filter connectors and hoses. This support device is to be installed so that the filter may be easily removed from the incubator and placed in the roof space near the incubator door to allow the filter to be easily removed when the door is opened.

The invention includes a second configuration to produce a similar system for the shield placement in the incubator. This configuration of the air purification system is used when space in the incubator for the filter system is a problem or restriction. The second configuration contains a motor box which is attached to the interior of the incubator through an access hole. This configuration may be used in incubators that do not have enough head space to accommodate the shield technology. The motor of the filter assembly is powered through a hole placed in the roof of the incubator or by accessing power through an access hole, which most incubators may have built into the side or rear wall of the incubator.

### BRIEF DESCRIPTION OF THE DRAWINGS

These and other objects and advantages of the invention will become more readily apparent from the following detailed description of several embodiments of the invention when taken in conjunction with the accompanying drawings in which:

FIG. 1 is a front view of an incubator with the door open and showing the adjunct filter system module disposed inside of the incubator attached to the roof of the incubator; and

FIG. 2 is a fragmented side sectional view of the roof of the incubator showing the plate and incubator assembly which is used to attach the filter assembly to the roof of the incubator.

### DETAILED DESCRIPTION OF SPECIFIC EMBODIMENTS OF THE INVENTION

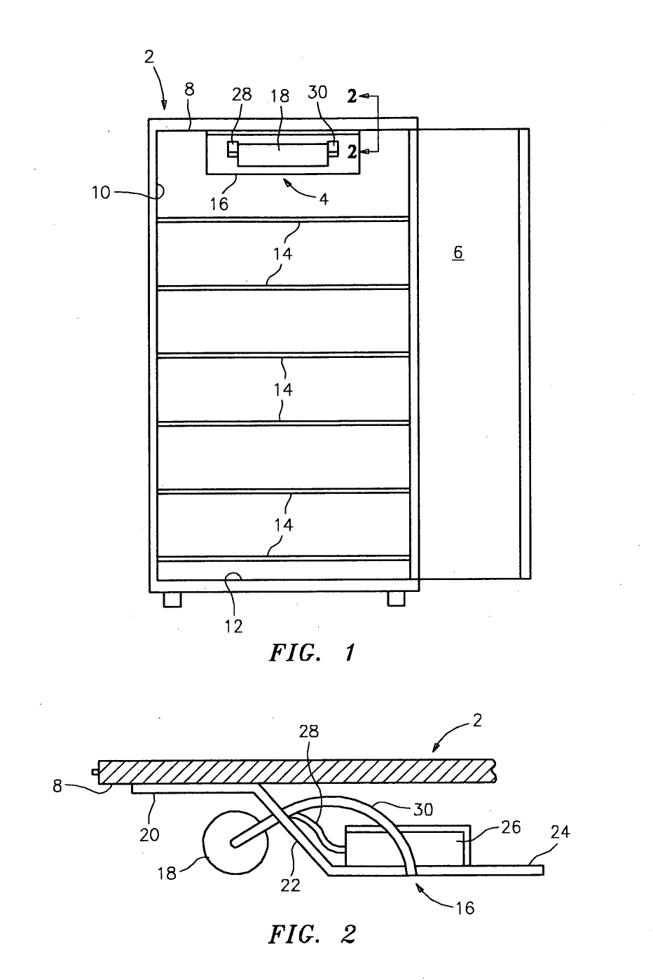

FIG. 1 is a front elevational view of an incubator 2 which has been retro-equipped with the air filter assembly 4 that is formed in accordance with the invention. The door 6 of the incubator 2 is shown in its open position. The incubator 2 includes an upper wall 8, side walls 10 and a bottom wall 12. A plurality of shelves 14 are disposed in the incubator 2. The shelves are for holding culturing trays which contain biological specimens, such as oocytes, embryos, sperm, or the like which are being treated in the incubator 2.

It is highly desirable to retrofit an air filter into the incubator 2, but a problem relating to space for the filter exists in incubators of the type shown in FIG. 1, which are very common specimen culturing incubators. We have devised a mounting system which can be used to mount an extant filter system known as the CODA filter system in the incubator 2 shown in FIG. 1. As noted above, the CODA air filter installation system is denoted generally by the numeral 4. The system 4 includes a mounting plate 16 which is affixed to the upper wall 8 of the incubator 2. The filter 18 is mounted on the plate 16.

Details of the plate 16 and manner in which the filter 18 is mounted thereon are shown in FIG. 2. As noted in FIG. 2, the plate 16 has a first horizontal part 20 which is secured to the roof or ceiling 8 of the incubator 2. The plate 16 has an intermediate downwardly angled part 22 on which the filter 18 is mounted. The plate further includes a second horizontal part 24 which is spaced apart from the roof 8 of the incubator 2. An air pump or fan 36 is mounted on the part 24 of the plate 16. Air transfer tubes 28 and 30 are connected to the filter 18. The air transfer tube 30 opens through the plate part 24 into the interior of the incubator 2 to draw air from the incubator 2 into the filter 18. The tube 28 interconnects the filter 18 with the pump 26. Thus the pump 26 effectively pumps air from the incubator 2 through the tube 30, through the filter 18, through the tube 28, and then back into the incubator 2.

When the filter 18 is mounted on the plate 16 in the manner described and shown, the filter 18 can be easily accessed through the open door 6 and can be thus easily changed when needed. Thus the plate 16 allows a filter 18 to be easily mounted and serviced in an incubator which presently has no air filter and thus the recirculated air in the incubator 2 can be kept free of contaminants from ambient surroundings.

It will be appreciated that the mounting system for the improved filters system for extant incubators is very simple to use, allows easy access to the filters for changing them in a timely manner, and will improve the performance of the incubators in question.

What is claimed is:

1. A tissue culturing incubator assembly comprising:
a) an incubator housing having a top panel, opposite side panels, a bottom panel, a back panel, a door opposite said back panel, said door being hinged to one of said side panels, and one or more shelves extending between said side panels for holding tissue specimens which are cultured in said incubator assembly;
b) a plate mounted on an internal surface of said top panel, said plate having:
i) a first part that is parallel to and secured to said top panel; and
ii) a second part that extends downwardly from said first part toward said bottom panel;
c) a filter mounted on said second part, said filter being exposed in said assembly and facing said door for easy access through said door; and
d) means for circulating air in the interior of said assembly through said filter and then returning the filtered circulated air to the interior of said assembly.

2. The assembly of claim 1 wherein said means for circulating is mounted on said plate.

3. The assembly of claim 1 wherein said means for circulating is mounted on a third part of said plate, which third part is spaced downwardly apart from said incubator top panel and parallel thereto.

4. An air circulating and filtering system for use in a tissue culturing incubator, said system comprising:
a) a mounting plate having first and third parts which are parallel to each other and vertically spaced apart from each other, said plate also having a second part which depends from said first part and interconnects said first and third parts;
b) a filter assembly mounted on said second part of said mounting plate, said filter assembly being disposed on said second part and facing away from said third part; and
c) means for circulating air through said filter assembly, said means for circulating air being mounted on said third part of said mounting plate.
